(19) United States
(12) Patent Application Publication
(10) Pub. No.: US 2007/0081985 A1
(43) Pub. Date: Apr. 12, 2007

(54) Lysozyme fortified milk and milk products

(75) Inventors: Stefano Ferrari, Rhinebeck, NY (US); Seth Feuerstein, Woodbridge, CT (US)

Correspondence Address: Norris, McLaughlin & Marcus P.A., 18th Floor, 875 Third Avenue, New York, NY, 10022, US

(73) Assignee: Saint Simeon LDA, Rhinebeck, NY (US)

(21) Appl. No.: 11/244,995

(22) Filed: Oct. 6, 2005

(51) Int. Cl. A61K 38/47; A61K 47/00; A61K 35/20

(52) U.S. Cl. 424/94.61; 424/535; 424/439

(57) ABSTRACT

Milk and/or yogurt fortified with lysozyme for augmentation of the immune system and aid the immune compromised.

### BACKGROUND OF THE INVENTION

1. Field of the Invention

The present invention relates generally to milk and milk products such as yogurt fortified with lysozyme

2. Description of Related Art

It is of growing concern that people, especially persons with immunity-compromised systems show due care with foods they consume. Dairy products, often consumed in large quantities by the young, often contain lower levels of anti-infective proteins and compounds than maternal milk. For instance, pediatricians widely recommend mothers to breast-feed their children when they are infants if possible, wherein one of the benefits is an improved infection profile. Historically, children were often fed milk products such as breast milk until 3-4 years old. Today however, children rarely consume such large quantities of such milk into those years. In addition, the elderly, as with the young, have immune systems which are compromised. The elderly are more susceptible to afflictions that come with increasing age. Infants have immune systems that are still developing and maturing. In fact, a study has shown food poisoning in infant food wherein strains of Bacillus licheniformis were found. (See Salkinoja-Salonen, M. S. et al., Toxigenic Strains of Bacillus licheniformis Related to Food Poisoning, Applied and Environmental Microbiology, October 1999, p. 4637-4645, Vol. 65, No. 10). Breastmilk contains many of the nutrients required for development and augmentation of the immature immune system in infants. It also contains non-nutritional components that may promote infant health, growth and development, such as antimicrobial factors, digestive enzymes, hormones, trophic factors, and growth modulators. However, it is not always possible to obtain human milk products, such as breastmilk, for those people whose immune system is compromised. In addition, liquid food such as breastmilk may not always be available or be optimal for the circumstances. On the other hand, solid food is not necessarily appropriate for infants and the elderly, because they may not be able to chew the food properly and/or may choke on the food causing potential digestive problems. Therefore, there is a need to protect people whose immune system can benefit from supplementing their diet by producing, for example, food which contains elements which are helpful to combat bacteria, virus, fungi, or other microbial particles which reside in the food. At the same time, there is a need to produce food which is more easily consumable by people such as infants and the elderly discloses a synthetic infant formula composition based on human milk proteins or host resistance factors, wherein one of the human milk proteins is lysozyme. However, both U.S. Pat. No. 6,270,827 and U.S. Pat. No. 6,020,015 are limited to infant formula. This formula is typically consumed until the end of an infants' first year when they often switch to milk and yogurt products purchased in markets after the first year.

Lysozyme is well known as an effective immunological agent. It is widely used in human therapy for the treatment of viral and bacterial infections. For example, U.S. Pat. No. 5,041,236 discloses an antibacterial composition using a ruminant stomach lysozyme. A further example is found in U.S. Pat. No. 4,355,022, which discloses an antibacterial solution comprising lysozyme for the oral cavity. Lysozyme is found in a large number of animal fluids, such as tears, pleural fluid, saliva, human milk and blood serum, as well as in a variety of organs such as the kidneys and lungs. In human milk, for example, lysozyme is present in a concentration ranging from about 0.12 g/L to about 0.5 g/L. Lysozyme is defined as a 1,4-beta-N-acetylmuramidase which cleaves the glycoside bond between the C-1 of N-acetyl-muramic acid and C-4 of N-acetylglucosamine in the peptidoglycan of bacteria (See Phillips, D. C., Sci. Am., 1966, 215: 78-90). The protective role of lysozyme has been observed to include lysis of microbial cell walls, adjuvant activity of the end products of peptidoglycan lysis, direct immunomodulating effects on leukocytes, and neutralization of bacterial endotoxins. Lysozyme is effective against gram positive and gram negative bacteria, as well as some types of yeasts. In this capacity, lysozyme can function as a broad spectrum antimicrobial agent. (See generally Biggar, W. D. and Sturgess, J. M., Infect. Immunol., 1977, 16: 974-982); Thacore, M. and Willet, H. P., Am. Rev. Resp. Diseases, 1966, 93: 786-790)). The antimicrobial effects of lysozyme often act synergistically with other defense molecules, including immunoglobulin and lactoferrin (See Jorieux et al., Characterization of Lactotransferrin Complexes in Human Milk, in Protides of the Biological Fluids, Proc. 32^{nd} Coll., 1984).

Because lysozyme is a natural constituent of the hen egg white, as well as milk in all mammals, it is viewed as completely harmless as an ingredient in food. The current commercial source for lysozyme is from hen egg whites. The human and hen egg white forms of lysozyme have 60% sequence similarity, but have a very similar 3-dimemsional structure. Human and hen egg white lysozymes differs in the amino acid sequence by 51 of 129 residues with one insertion at the position between 47 and 48 in the hen lysozyme (See Mine, Shouhei et al. Analysis of the internal motion of free and ligand-bound human lysozyme by use of ¹⁵N NMR relaxation measurement: A comparison with those of hen lysozyme, 2000, Protein Science 9: 1669-1684). Recently, it was disclosed in U.S. 2004-0111766 A1 a process for isolating a recombinant form of human lysozyme from a transgenic rice plant. In fact, U.S. Pat. No. 6,270,827 employs human lysozyme due to the alleged side effects due to a body's potential immune response when hen egg white lysozyme is used for medical purposes. However, studies have shown that immunogenic reactions to the hen egg white form of lysozyme, if any, are minor. Antibody reactions have been seen to other protein components in the egg white, but rarely to lysozyme. In addition, other lysozymes, such as from an equine animal and cow are not derived from potentially allergenic sources and can be used as described herein (See e.g., Langeland, T. & K. Aas, Allergy to Hen's Egg White; Clinical and Immunological aspects. In: Brostoff, J. & S. J. Challacombe (eds.) Food Allergy and Intolerance, London, Bailliere Tindall, 1987, pgs. 367-374; See also Godovac-Zimmermann J, Conti A, Napolitano L., The primary structure of donkey (Equus asinus) lysozyme contains the Ca(II) binding site of alpha-lactalbumin, Biol Chem Hoppe Seyler, 1988, 369:1109-15; See also Godovac-Zimmerman et al., Biol. Chem. Hoppe Seyler, 1987, 368: 427-433). Lysozyme has been used to study the bacterium Bifidus bacillum which is said to contribute to a healthy flora and aid in digestion (Nishihava, K. and Isoda, K., Fecal bacterial flora in the use of lysozyme treated dried milk, 1967, Acta Paediatr. Jpn., 71, 95). In the study, lysozyme was added to infant dry milk to simulate human milk, and found lysozyme contributed to an increase in the number of B. bacillum in the intestine of infants. U.S. Pat. No. 6,020,015 and U.S. Pat. No. 6,270,827 disclose synthetic infant formula compositions based on human milk proteins. However, both U.S. Pat. No. 6,270,827 and U.S. Pat. No. 6,020,015 are limited to solid and/or liquid forms. Both U.S. Pat. No. 6,270,827 and U.S. Pat. No. 6,020,015 disclose an infant formula in liquid or concentrate form. In addition, they do not disclose lysozyme in combination with trypsin or from other sources such as donkey and cow. The isolated lysozyme in U.S. 2004-0111766 A1 discloses use in food, but again, the lysozyme is for use in solid or liquid form. Hen egg white lysozyme is employed in GB 2 379 166 A, which discloses animal feed. The animal feed is for monogastric and/or non-ruminant animals such as poultry, pigs, piglets, calves and fish. There is no disclosure of the use of a human recombinant lysozyme or of the animal feed containing lysozyme in combination with lactoferrin and/or trypsin. U.S. 2005-0100634 A1 discloses human milk proteins for use in nutritional formulations. However, there is no disclosure of a combination with trypsin and the use of lysozyme from non-human sources.

Therefore, there is a need for milk and/or yogurt products fortified with lysozyme, especially for those people whose immunity systems are compromised, without the foregoing disadvantages. The present invention solves the deficiencies stated in the prior art, while providing improvements as stated herein.

### SUMMARY OF THE INVENTION

The present invention relates generally to milk and/or yogurt products fortified with lysozyme.

### DETAILED DESCRIPTION OF THE INVENTION

Lysozymes act as enzymes that cleave peptidoglycans, a ubiquitous cell wall component of microorganisms, in particular bacteria. Gram-positive bacteria are highly susceptible to lysozyme due to the polypeptidoglycan on the outside of the cell wall. Gram-negative strains have a single polypeptidoglycan layer covered by lipopolysaccharides and are therefore less susceptible to lysis by lysozyme.

Lysozyme from human and non-human sources is contemplated. In one example, U.S. 2004-011766, which is hereby incorporated by reference, discloses human recombinant lysozyme which is expressed in rice. In another example, isolated human lysozyme is disclosed in U.S. Pat. No. 5,618,712, which is hereby incorporated by reference. Lysozyme has been isolated and/or reported in non-human sources ranging from the hen egg white (U.S. Pat. No. 3,515,643, which is hereby incorporated by reference) to the ficus plant (See Meyer, K. et al, Lysozyme of Plant Origin, J. of Biol. Chem., 1946, Vol. 163, Pages 733-740) to the Asterias rubens, or common starfish (Bachali, Sana, et al., The lysozyme of the starfish Asterias rubens, 2004, Eur. J. Biochem., Vol. 271, Pages 237-242). For example, donkey lysozyme is a c-type lysozyme which is 129 amino acids long. It exhibits 50% homology to the human protein. (See Godovac-Zimmermann J, Conti A, Napolitano L., The primary structure of donkey (Equus asinus) lysozyme contains the Ca(II) binding site of alpha-lactalbumin, 1988, Biol Chem Hoppe Seyler, 369 (10): 1109-15). Genetic variants of donkey lysozyme have been established. (See Herrouin, Maryse et al., New Genetic Variants Identified in Donkey's Milk Whey Proteins, 2000, J. Protein Chem, 19: 105-115.) Other methods exist for the isolation of such proteins. For instance, Maullu et al. use whey and cottage cheese as culture media for strains of Kluyveromyses lactis transformed with the human lysozyme gene and generate considerable amounts of recombinant protein (Maullu et al., Journal of Applied Microbiology, 1999, 86: 182-186).

Lactoferrin comprises a protein found naturally within biological fluids, such as milk and saliva, at mucosal surfaces and within white blood cells. It is thought that lactoferrin has anti-bacterial properties, while still protecting the body. Lactoferrin and lysozyme in human breast milk has been shown to act in complement to prevent gastroenteritis and allergies (See Francis D., in Infant Nutrition, Birch, G. G. and Parker, K. J., Eds., Applied Science Publishers, London, 1980; See also Carlsson, B., Cruz, J. R., Garcia, B., Hanson, L. A., and Urrutia, J. J., Immune facors in human milk, Nutrition and Metabolism of the Fetus and Infant, Visser, H K A., Ed., Martinus Nijhoff, The Hague, 1979, 263). In addition, lactoferrin appears to effectively kill a range of fungi and yeasts, including the causative agent of thrush, Candida albicans. Moreover, research has shown that lactoferrin can prevent viruses, such as HIV, hepatitis and CMV, from binding to the body's cells and therefore prevents viral infection.

Lactoferrin is one of the principle proteins responsible for providing protection to infant mammals before their immune systems begin to function. It is a minor protein in cow's milk (0.3% by weight) and is extracted from skim milk or whey through protein separation. Apart from milk, lactoferrin is generally produced and released in the body in the digestive, respiratory and reproductive systems through bodily secretions such as saliva, tears, and nasal secretions. Lactoferrin is also produced by a special group of white blood cells known as neutrophils.

Lactoferrin occurs naturally in three forms: (i) iron-saturated, (ii) iron-free, and (iii) immobilized (Activated). It is thought that the iron-free and immobilized forms of lactoferrin have the highest antimicrobial abilities through the binding of iron required by bacteria for growth and the ability for lactoferrin to detach bacteria from surfaces and eliminate bacterial attachment structures.

In a preferred embodiment, the milk and/or yogurt is fortified with lysozyme and/or lysozyme in combination with lactoferrin and/or trypsin. The term “fortified” as used herein means 1 to 4 times the concentration normally found in human breastmilk. “Young children” as used herein comprises children that are between the age of over 3 years old to less than 13 years old. Trypsin is a digestive enzyme produced in the pancreas to digest proteins. It has been used in treatments for wounds and for diabetes. It has also been used in food processing in infant formulas to aid in digestion. In one preferred embodiment, the milk and/or yogurt is fortified with both lysozyme and trypsin. In another preferred embodiment, the milk and/or yogurt is fortified with lysozyme, lactoferrin and trypsin. In a preferred embodiment, the lysozyme is either isolated from the hen egg white, donkey milk, cow's milk, goat's milk, cauliflower or in the human recombinant form. In another preferred embodiment, the lysozyme is an isolated milk lysozyme.

The milk and/or yogurt can be any product from those categories generally consumed by infants, geriatric patients or persons whose immune systems are compromised such as HIV patients, cancer patients and transplant patients. Milk and/or yogurt products include milk, shakes, milk powders, yogurt, and other dairy products. In one preferred embodiment, milk and/or yogurt fortified with lysozyme has a content of lysozyme which is about 0.25 g/L to about 2.0 g/L, and more preferably about 1.0 g/L to about 2.0 g/L.

It should be understood that the preceding is merely a detailed description of one preferred embodiment or a small number of preferred embodiments of the present invention and that numerous changes to the disclosed embodiments can be made in accordance with the disclosure herein without departing from the spirit or scope of the invention. The preceding description, therefore, is not meant to limit the scope of the invention in any respect. Rather, the scope of the invention is to be determined only by the issued claims and their equivalents.

1. Milk and/or yogurt product fortified with lysozyme and suitable for human consumption.

2. The milk and/or yogurt according to claim 1, which comprises lactoferrin and/or trypsin.

3. The milk and/or yogurt according to claim 1, wherein the lysozyme is a human recombinant lysozyme.

4. The milk and/or yogurt according to claim 1, wherein the lysozyme is a non-human lysozyme.

5. The milk and/or yogurt according to claim 3, wherein the human recombinant lysozyme is a milk lysozyme.

6. The milk and/or yogurt according to claim 4, wherein the non-human lysozyme is a milk lysozyme.

7. The milk and/or yogurt according to claim 4, wherein the non-human lysozyme is a hen egg white lysozyme.

8. The milk and/or yogurt according to claim 4, wherein the non-human lysozyme is an equine lysozyme.

9. The milk and/or yogurt according to claim 8, wherein the equine lysozyme is an isolated donkey lysozyme.

10. The milk and/or yogurt according to claim 1, wherein the lysozyme is at a concentration of about 0.25 to about 2.0 g/L.

11. The milk and/or yogurt according to claim 10, wherein the lysozyrne is at a concentration of about 1.0 to about 2.0 g/L.

12.-14. (canceled)

15. The milk and/or yogurt according to claim 1, which is formulated with other food stuffs.

16. (canceled)

17. A method of protecting against infections due to food poisoning, said method comprising administering to a patient in need thereof an effective amount therefor of a milk and/or yogurt according to claim 1.

18. The method according to claim 17, wherein the patient has a compromised immune system.

19. The method according to claim 17, wherein the patient is an infant, toddler or young child.

20. The method according to claim 17, wherein the patient is a geriatric patient.

21. The method according to claim 17, wherein the patient is a diabetic.

22. The milk and/or yogurt according to claim 1, which is milk fortified with lysozyme.

23. The milk and/or yogurt according to claim 1, which is yogurt fortified with lysozyme.
